(19) United States
(12) Patent Application Publication
(10) Pub. No.: US 2008/0119386 A1
(43) Pub. Date: May 22, 2008

(54) Nutritional formula for athletes' recovery

(76) Inventor: Carl Germano, New City, NY (US)

Correspondence Address: SCHWEITZER CORNMAN GROSS & BONDELL LLP, 292 MADISON AVENUE - 19th FLOOR, NEW YORK, NY, 10017, US

(21) Appl. No.: 11/603,482

(22) Filed: Nov. 22, 2006

(51) Int. Cl. A61K 38/00; A61K 31/122; A61P 21/00

(52) U.S. Cl. 514/2; 514/688

(57) ABSTRACT

A nutritional composition for athletes comprising in combination components for reducing oxidative stress, components for energy production and for maintaining lean muscle, the key component of which is SOD/gliadin.

Various products, such as ready-to-drink formulas, are found in health food stores and gyms to cater to the strains put on the body due to severe exercise. Most of these products are not nutritionally complete and have limited health benefits or act as a “single magic bullet” by only addressing one of the several issues arising in the athlete's body.

Thus, there is a need for a formulation, preferably in the form of a nutritional beverage, that addresses the major nutritional concerns of the athlete, and particularly the recovery after strenuous activity. The present composition serves to satisfy this requirement.

### BRIEF DESCRIPTION OF THE INVENTION

The present composition is a nutritional adjunct to an exercise program to assist in the recovery process in order to improve performance. It addresses the major nutritional concerns of the athlete, which include reducing muscle breakdown, reducing oxidation stress, reducing lactic acid buildup, supporting energy production, reducing fatigue, and providing immune response to the stressors associated with rigorous exercise. It will normally take the form of a delicious multi-action, multi-component, nutritional powdered mixture which can be put into water or a juice mixture. The mixture can then be incorporated in solid forms such as ice pops, bars or other solids subject to be taken orally. As an alternative to stimulants (caffeine, ephedra, guarana, herbe mate, citrus aurantium, etc.) or excessive dosing of protein, this sports oriented compositional nutritional formula was developed to address the multiple nutritional needs of athletes (reduction of oxidative stress, support of mitochondrial function (fatigue reduction), building of muscle (lean tissue) and support of immune function). In efforts to improve performance and strength through proper recovery, the compositional formula represents a clear alternative to the typical products found in the marketplace.

The present composition is a high protein, complex carbohydrate, low sugar nutritional recovery/performance drink mix that contains a comprehensive blend of specialized nutrients that play major roles in addressing the above-mentioned needs that have been identified in athletes. It has been developed to meet the NCAA guidelines for nutritional products for the collegiate marketplace and is part of the Banned Substance Control Group's certification program which certifies the formula is free of banned substances for the professional and collegiate marketplace. The roles of each of the components are as follows:

To address oxidative stress/lactic acid build-up and help assist in reducing oxidative stress and lactic acid, the role of SOD/gliadin, CoQ10, and standardized high ORAC fruit extracts is particularly important.

To address maintenance of lean muscle and help assist in reducing muscle breakdown, the role of whey protein and nucleotides is particularly important.

To address fatigue and energy loss and help assist in increasing energy, the role of CoQ10, ribose, nucleotides and MCT's is particularly important.

To address lowered immune response and help assist in supporting immune response, the role of whey protein, adequate calories, vitamins/minerals and beta glucans is particularly important.

Each of the proceeding nutritional needs of the athlete and the effect of the components of the present composition in overcoming these needs are discussed below.

### DETAILED DESCRIPTION OF THE INVENTION

1. Reducing Oxidative Stress—Oxygen consumption greatly increases during exercise, which leads to increased free radical production. The body counters the increase in free radical production through the antioxidant defense system. When free radical production exceeds clearance, oxidative damage occurs. Free radicals formed during chronic exercise may exceed the protective capacity of the antioxidant defense system in the body, thereby making the body more receptive to disease and injury. A free radical attack on a membrane usually damages a cell to the point that it must be removed by the immune system. If free radical formation and attack are not controlled within the muscle during exercise, a large quantity of muscle could easily be damaged. Damaged muscle could in turn inhibit performance by the induction of fatigue from destructive by-products.

While many of the nutrients in the present composition play a role in antioxidant support, the roles of SOD/gliadin, CoQ10, and standardized high ORAC fruit extracts are most critical to this issue. In addition to a comprehensive base of nutriceutical ingredients, the present composition contains the first orally effective vegetarian form of super oxide dismutase—SOD/gliadin. It serves as a major antioxidant defense against oxygen-derived free radicals that are abundant after strenuous activity.

Orally administrable super oxide dismutase (SOD) in combination with a lipid or protein derived from plants is described in U.S. Pat. No. 6,045,809 whose specification is hereby incorporated by reference. The protein can be a Prolamine derived from different cereals. Particularly preferred in the present invention is SOD/gliadin using gliadin derived from wheat. SOD/gliadin is sold under the trademark “GliSODin” by Akryon S.a.r.l. of Neuilly Sur Seine, France.

As an antioxidant, SOD helps to decrease oxidative stress—it is the master cellular defense enzyme of the cell and serves as significant support for the immune system. It can counter the harmful effects of free radicals, thereby diminishing their negative effects on the body. In addition, GliSODin® has been shown in reduce oxidative stress, upregulate antioxidant defense, reduce lactic acid and exert a positive influence on immune function.

In addition to SOD/gliadin, the present composition also contains a blend of other ingredients that help support the cells' ability to defend against oxidation stress.

Whey Protein—whey has been shown to assist in cellular defense by increasing available glutathione—another important cellular antioxidant required by the body to ward off the effects of oxidative stress.

Coenzyme Q10—a powerful cellular antioxidant with roles in preventing oxidation of fatty cell membranes and ultimately cell death. Coenzyme Q10 is an essential component of cellular energy production and respiration by participation in the mitochondrial electron transport system, which supplies energy (ATP) for a variety of physiological functions. Virtually every cell of the human body contains coenzyme Q10.

High ORAC Fruit Antioxidants—contain important accessory antioxidants naturally found in high antioxidant containing fruits.

Examples of suitable fiber fruit antioxidants are wild blueberry extract, grape seed extract powder, raspberry seed extract powder, cranberry powder, prune powder, tart cherry powder, wild bilberry extract, and strawberry powder.

A high ORAC Score indicates a high total antioxidant capacity.

2. Components for Increasing Energy Production—Inability of the cells to produce adequate and optimum energy results from the disruption of the energy producing part of the cells called mitochondria. Mitochondria are tiny organelles found inside all human cells of the body. Essentially, they are the cell's “power plants” that produce energy (ATP) as well as being involved in protein and fat processing. The term “ATP” denotes adenosine triphosphate. Damage done to the mitochondria are typically attributed to the buildup of free radicals and other noxious by-products such as lactic acid that accumulates during and after strenuous activity. Neutralizing these damaging waste products and clearing them from the cell is critical to optimizing performance. The present invention contains a patented blend of ingredients that help support the function of the mitochondria and assist in the production of energy in the cell. These ingredients include:

SOD/gliadin—this unique form of super oxide dismutase has been shown to protect mitochondrial DNA from oxidative stress in humans as well as lower lactic acid buildup.

Nucleotides—important nutrient compounds that are involved in almost all cellular processes and play a major role in structural, metabolic, energetic and regulatory functions. They make up the basic framework material required for muscle synthesis and is required for growth and cell division. Adenosine triphosphate (ATP), a nucleotide, is the major source of chemical energy used in metabolism, driving almost all cellular processes.

Coenzyme Q10 (COQ10)—is an essential component of cellular energy production and respiration since it participates in the mitochondrial electron transport system, which supplies energy (ATP) for a variety of physiological functions. Muscle mitochondria lack adequate COQ10 in several chronic conditions.

Ribose—is a carbohydrate used by the cells to form a primary source of all the body's energy—ATP. Ribose plays a key role in the generation and recovery of ATP by assisting in regenerating the adenosine portion of the ATP molecule. Ribose can offer powerful complimentary support to other nutrients addressing energy depletion by aiding normal ATP production.

3. Maintaining Lean Muscle—Maintenance of lean muscle is of utmost importance in the athlete. A common problem during strenuous activity is lean muscle loss or wasting as muscle tends to break down after strenuous activity. This problem can have a serious impact on performance and recovery. Several nutritional issues related to the loss of muscle during activity include: inadequate caloric (carbohydrate and protein) intake, problems with metabolism and elevations of inflammatory compounds that break down muscle (certain cytokines). Nutritional supplementation has played an important role in boosting caloric intake but increasing calories may not be the only answer. The present composition contains a blend of ingredients that help maintain lean tissue by providing high quality protein and compounds that assist in building muscle and prevent its breakdown. Some of these ingredients include:

Nucleotides—Are naturally occurring compounds that are involved in key metabolic processes, including energy metabolism and enzymatic reactions. They are building blocks of (DNA) and (RNA) in muscle tissue and a dietary source is required to promote optimal tissue growth. Nucleotides have been commonly used in post trauma as a means to rebuild muscle and support immune function.

Whey—When it comes to nutritional support directed at maintaining lean mass, adequate calories and good quality protein are essential. By virtue of its biological value (BV), whey protein has long been considered the best source of protein for building and retaining lean muscle mass.

As will be noted from the above discussion, whey proteins also serve as antioxidants, while nucleotides also serve as energy enhancers.

4. Supporting Immune System—Essentially good nutritional support supplying essential macronutrients and micronutrients are critically important for maintaining a proper immune system. Nevertheless, if the cells of the immune system cannot produce energy efficiently (mitochondrial dysfunction), have poor antioxidant defenses (oxidative stress) and the body is losing important lean muscle (muscle breakdown), immune support cannot effectively be achieved.

While exercise has been shown to have a positive effect on the immune system, over-training or excess exercise (such as a marathon) suppresses immunity anywhere from several hours to a week or longer, thus creating periods of vulnerability to upper respiratory tract infection or other health problems.

The present composition addresses the foregoing needs (antioxidant defenses, lean mass preservation and energy production) and helps support the athlete's immune system.

The range of components in the compositions of the present invention are set forth in Table A below.

All values are on a daily basis. “U” stands for units of activity.

TABLE A
Component Ratios

| | Broad Range | Preferred Range |
|---|---|---|
| To address oxidative | | |
| stress/lactic acid | | |
| SOD/gliadin | 5 IU-1,000 IU | 500 IU-1,000 IU |
| CoQ10 | 5 mg-300 mg | 75 mg-150 mg |
| Standardized high | 5 mg-600 mg | 150 mg-300 mg |
| ORAC fruit extracts | | |
| To address maintenance | | |
| of lean muscle | | |
| Whey protein | 1 g-50 g | 20 g-40 g |
| Nucleotides | 5 mg-1,000 mg | 300 mg-600 mg |
| To address fatigue and energy loss | | |
| CoQ10 | 5 mg-300 mg | 75 mg-150 mg |
| Ribose | 5 mg-6,000 mg | 1,000 mg-3,000 mg |
| Nucleotides | 5 mg-1,000 mg | 300 mg-600 mg |
| Medium chain triglycerides | 1 g-20 g | 4 g-8 g |
| (MCTs) | | |

Further descriptions of the above components will be found in U.S. Pat. No. 6,503,506 by the instant inventor and are incorporated herein by reference.

### EXAMPLE

A nutritional blend in accordance with the invention, consisting of 75 mg CQ10, 500 U SOD/gliadin, 300 mg L-Carnitine, 100 mg nucleotides, 750 mg d-ribose, 100 mg beta glucans, and 12.5 mg fruit polyphenols was formulated as a liquid drink. A double blind placebo control trial at Rutgers University Division 1 soccer was performed, in which the drink was administered daily to a group (the “formulation group”) of players over a 20-day preseason training schedule. A second control group received an isocaloric equivalent. Lactic acid threshold, oxidative stress markers (LPO's, isoprostanes) and a progressive maximal treadmill test measured and given at the start and conclusion of the training schedule revealed significant changes in performance capacity. Significant results included:
Significant changes in performance capacity and reduced oxidative stress in the formulation group as compared to the control group.
Improved lactic acid responses to exercise and time to exhaustion for the formulation group as compared to the control.
Significantly lower oxidative stress markers (LPOs and isoprostanes) in the formulation group as compared to the control.

An additional double blind placebo controlled study was made with Rutgers' Division 1 football athletes. The effect of the present composition on strength, body composition and recovery markers/oxidative stress was evaluated. The athletes received either the formulation in the drink form or an isocaloric equivalent on a daily basis over a 7-week training schedule. Body composition, Wingate Test and recovery markers were tested at the start and finish of the training.

Over the course of training, the following was observed:
Power/Strength Wingate Testing: Peak power was measured for each test and standardized by body weight. The increase in peak power in the formulation group, though, was approximately 86% greater than the increase in the control group.
Recovery Markers—the formulation group had significantly improved testosterone/cortisol ratios as compared to the control group.
Body Composition With similar increases in lean body mass, the control group had an increase in % body fat while the formulation group had a decrease in % body fat. There was an increase in fat mass in the control group while those in the experimental group had a decrease in fat mass (−1.6 lbs).

The above tests illustrate the advantages of the present nutritional formulation to address major nutritional concerns of the athlete. The present composition serves as a modular component system that provides support in addressing all the foregoing needs and issues of the athletes, as well as helping support the athlete's immune system.

I claim:

1. A nutritional composition for athletes comprising in combination:
(i) components for reducing oxidative stress comprising SOD/gliadin;
(ii) components for energy production; and
(iii) components for maintaining lean muscle.

2. The composition of claim 1, wherein the components for reducing oxidative stress further comprises whey protein, Coenzyme Q10 and high ORAC fruit antioxidants.

3. The composition of claim 1, wherein the components for energy production are selected from the group consisting of SOD/gliadin, nucleotides, Coenzyme Q10 and Rebose.

4. The composition of claim 1, wherein the components for maintaining lean muscle are selected from the group consisting of nucleotides and whey protein.

5. The composition of claim 1 which comprises: 5 IU-1,000 IU of SOD/gliadin as component (i); 5 mg-300 mg of Coenzyme Q10; 5 mg-6,000 mg of Ribose and 5 mg-1,000 mg of nucleotides as component (ii) and 1 g-50 g of whey protein and 5 mg-1,000 mg of nucleotides as component (iii).

6. The composition of claim 1, wherein SOD/gliadin serves as a component for reducing antioxidant stress as well as a component for energy production.

7. The composition of claim 1, wherein Coenzyme Q10 serves as a component for reducing oxidative stress and as a component for energy production.

8. The composition of claim 1, wherein whey protein serves as a component for reducing oxidative stress and as a component for maintaining lean muscle.

9. A nutritional composition for athletes comprising in combination:
(i) components for reducing oxidative stress selected from the group consisting of 5-1,000 IU of SOD/gliadin, 5-300 mg Coenzyme Q10 and 5-600 mg high ORAC fruit antioxidants;
(ii) components for increasing energy production selected from the group consisting of SOD/gliadin, 5-1,000 mg nucleotides, 5-300 mg Coenzyme Q10 and 5-6,000 mg Ribose; and
(iii) components for maintaining lean muscle selected from the group consisting of 5-1,000 mg nucleotides and 1-50 g whey protein.
